SEQUENCE LISTING (2 sequences)

SEQ ID NO: 1 - RNA, 20 nt, synthetic construct
  source 1..20: mol_type = other RNA
ccgtcccctggagaagaaca

SEQ ID NO: 2 - RNA, 20 nt, synthetic construct
  source 1..20: mol_type = other RNA
aggccccctcccaggcgtag

(19) United States
(12) Patent Application Publication
(10) Pub. No.: US 2026/0042859 A1
(43) Pub. Date: Feb. 12, 2026

(54) ENHANCEMENT OF PHAGOCYTOSIS

(71) Applicant: The Board of Trustees of the Leland Stanford Junior University, Stanford, CA (US)

(72) Inventors: Michael C. Bassik, Stanford, CA (US); Roarke A. Kamber, Stanford, CA (US); Mingxin Gu, Stanford, CA (US)

(21) Appl. No.: 19/109,191

(22) PCT Filed: Sep. 6, 2023
(86) PCT No.: PCT/US2023/073555

§ 371 Date: Mar. 6, 2025

Related U.S. Application Data
(60) Provisional application No. 63/374,690, filed on Sep. 6, 2022.

(51) Int. Cl. C07K 16/28; A61K 38/17; A61K 45/06; A61P 35/00; C12N 5/0786; C12N 5/0787

(52) U.S. Cl. CPC C07K 16/2887; A61K 38/1709; A61K 45/06; A61P 35/00; C07K 16/2896; C12N 5/0642; C12N 5/0645; C07K 2317/76

(57) ABSTRACT

The present disclosure provides methods for treating a disease or disorder or enhancing phagocytosis of a target cell. The methods comprise administering or contacting an immune cell with an inhibitor of paired immunoglobulin-like type 2 receptor-alpha (PILRA).

### CROSS-REFERENCE TO RELATED APPLICATIONS

This application claims the benefit of U.S. Provisional Application No. 63/374,690, filed Sep. 6, 2022, the content of which is herein incorporated by reference in its entirety.

### FIELD

The present disclosure provides methods for treating a disease or disorder (e.g., cancer and autoimmune disorders) and methods for enhancing phagocytosis of a target cell.

### Sequence Listing Statement

The content of the electronic sequence listing titled STDU2-41031.601.xml (Size: 2,839 bytes; and Date of Creation: Sep. 6, 2023) is herein incorporated by reference in its entirety.

### BACKGROUND

Macrophages, which are a type of white blood cells of the mononuclear phagocyte immune system, play vitally important roles in anti-infective immunity, the maintenance of tissue homeostasis, and the protection of a body through the functions of engulfing foreign substances through phagocytosis facilitating their breakdown and digestion. Macrophages also clear away harmful matter, including cellular debris and tumor cells in vivo. While monoclonal antibodies and CD47 blockade have been used as anti-cancer agents, in part by driving phagocytosis of tumor cells, existing therapies suffer from low response rates in patients.

### SUMMARY

Disclosed herein are methods of treating a disease or disorder. In some embodiments, the methods comprise administering to a subject in need thereof an inhibitor of paired immunoglobulin-like type 2 receptor-alpha (PILRA).

In some embodiments, the methods further comprise administering an inhibitor of at least one anti-phagocytic factor. In some embodiments, the at least one anti-phagocytic factor comprises small cell adhesion glycoprotein (SMAGP).

The methods may further comprise contacting the cell with at least one or both of a tumor antigen (TA)-targeting antibody and a CD47 blocking antibody. In some embodiments, the TA-targeting antibody is a CD20 blocking antibody. In some embodiments, the CD47 blocking antibody comprises an anti-CD47 antibody, an anti-SIRPalpha antibody, or any combination thereof.

The disease or disorder may comprise any disease or disorder in which cell elimination restoration of phagocytosis is the desired outcome, including, but not limited to: an autoimmune disorder, atherosclerosis, or cancer. In some embodiments, the disease or disorder is an autoimmune disorder, e.g., rheumatoid arthritis and multiple sclerosis.

In some embodiments, the disease or disorder is cancer. In some embodiments the cell is a cancer cell. The cancer or cancer cell may be any cancer. In some embodiments, the cancer or cancer cell is resistant to antibody-dependent cellular phagocytosis (ADCP). In some embodiments, the cancer or cancer cell overexpresses SMAGP. In some embodiments, the cancer or cancer cell is a solid tumor. In some embodiments, the cancer or cancer cell comprises lymphoma, cervical cancer, lung cancer (e.g., non-small-cell lung cancer and small-cell lung cancer), colorectal cancer, ovarian cancer, breast cancer and/or leukemia.

Also disclosed herein are methods of enhancing phagocytosis of a target cell. In some embodiments, the methods comprise contacting an immune cell with an inhibitor of PILRA. In some embodiments, the contacting comprises administration to a subject in need thereof.

In some embodiments, the immune cell comprises an innate immune cell. In some embodiments, the innate immune cell is a monocyte, a neutrophil, or a macrophage.

In some embodiments, the target cell overexpresses SMAGP. In some embodiments, the target cell is a cancer cell. In some embodiments, the cancer cell comprises lymphoma, cervical cancer, lung cancer, colorectal cancer, ovarian cancer, breast cancer and/or leukemia.

Further disclosed herein are compositions comprising an inhibitor of paired immunoglobulin-like type 2 receptor-alpha (PILRA) and an inhibitor of at least one anti-phagocytic factor. In some embodiments, the at least one anti-phagocytic factor comprises small cell adhesion glycoprotein (SMAGP).

In some embodiments, the compositions further comprise at least one or both of a tumor antigen (TA)-targeting antibody and a CD47 blocking antibody. In some embodiments, the TA-targeting antibody is a CD20 blocking antibody. In some embodiments, the CD47 blocking antibody comprises an anti-CD47 antibody, an anti-SIRPalpha antibody, or any combination thereof.

Other aspects and embodiments of the disclosure will be apparent in light of the following detailed description and accompanying figures.

### BRIEF DESCRIPTION OF FIGURES

FIGS. 1A-1B show SMAGP is a major regulator of phagocytosis resistance in RKO colon cancer cells. FIG. 1A is a schematic of the genome-wide CRISPR knockout screen conducted in RKO colon cancer cells for regulators of resistance to anti-CD47-driven macrophage phagocytosis.

FIG. 1B is a volcano plot of genome-wide knockout screen in RKO cells depicted in FIG. 1A.

FIG. 2 shows phagocytosis uptake of pHrodo-labeled Ramos dCas9-VPR cells expressing either SMAGP-targeting sgRNA or sgRNA targeting a Safe locus by primary human macrophages in the presence of anti-CD47 and anti-CD20 antibodies. The phagocytosis index was calculated as the total average pHrodo Red signal per well, normalized to signal in cells expressing Safe sgRNA at the 4h timepoint (n=3 cell culture wells).

FIGS. 3A-3C show the identification of PILRA as a candidate regulator of SMAGP-mediated resistance to phagocytosis. FIG. 3A is schematics of macrophage knockout screen, using a CRISPR knockout sub-library that is enriched for phagocytosis regulators and immune receptors in J774 macrophages, for uptake of calcein+control Ramos cells and Far-red+SMAGP-overexpressing Ramos cells. FIG. 3B is a volcano plot of all genes required for uptake of control Ramos cells (comparison 1 of screen depicted in FIG. 3A). FIG. 3C is a volcano plot of genes selectively required for uptake of SMAGP+ cells relative to control cells (comparison 2 of screen depicted in FIG. 3A).

FIG. 4 is phagocytosis assay uptake of pHrodo-labeled GFP-FLAG control or SMAGP-FLAG expressing Ramos cells by Safe knockout or PILRA knockout J774 cells in the presence of anti-CD47 and anti-CD20 antibodies. Data were normalized to signal in GPF-FLAG control Ramos cells fed to Safe knockout J774 cells at the 9h timepoint (n=3 cell culture wells).

FIG. 5 is phagocytosis assay uptake of pHrodo-labeled control, SMAGP+, and MUC1+Ramos cells by Safe knockout (PILRA+) or PILRA knockout (PILRA-) J774 cells in the presence of anti-CD47 and anti-CD20 antibodies. Data were normalized to signal in control (PILRA+) Ramos cells fed to Safe knockout J774 cells at the 9h timepoint (n=3 cell culture wells).

FIG. 6 is phagocytosis assay uptake of pHrodo-labeled Safe knockout or SMAGP knockout RKO cells by Safe knockout or PILRA knockout J774 cells in the presence of anti-CD47 antibody. Data were normalized to signal in Safe knockout control RKO cells fed to Safe knockout J774 cells at the 10h timepoint (n=3 cell culture wells).

FIG. 7 is phagocytosis assay uptake of pHrodo-labeled Ramos dCas9-VPR cells expressing Safe-targeting sgRNA or SMAGP-targeting sgRNA by Safe knockout or PILRA knockout J774 macrophages in the presence of anti-CD47 and anti-CD20 antibodies, with or without anti-mouse PILRA antibody. Data were normalized to signal in sgSafe control Ramos cells fed to sgSafe ko J774 cells without anti-mouse PILRA antibody at the 9h timepoint (n=3 cell culture wells).

FIG. 8 is a Western blot for non-transfected wildtype Ramos cells, and dCas9-VPR Ramos cells transduced with sgRNA targeting SMAGP (sgSMAGP) or non-functional safe domain (sgSAFE).

FIG. 9A is images of pHrodo-labelled SMAGP-FLAG overexpressing Ramos cells at 0 h and 9 h incubation with PILRa or Safe knockout J774 macrophages with anti-CD47 and anti-CD20 antibodies (error bar=100 μm). FIG. 9B is a graph of the results from a phagocytosis assay for uptake of pHrodo Red-labeled GFP-FLAG control or FLAG-SMAGP expressing Ramos cells by Safe knockout or PILRa knockout human primary derived macrophages in the presence of anti-CD47 and anti-CD20 antibodies. Data were normalized to signals in GPF-FLAG control Ramos cells fed to Safe knockout human primary macrophages at the 3h timepoint (n=3 cell culture wells).

FIGS. 10A-10E show T7A and T17A double mutations on SMAGP significantly inhibit SMAGP-mediated phagocytosis suppression and inhibit PILRa direct binding. FIG. 10A is a schematic representation of SMAGP protein. FIG. 10B is a schematic representation of PILRa protein. FIG. 10C shows expression of N-terminus FLAG-tagged wildtype and mutant SMAGP in wildtype Ramos cells by western blot with antibodies against SMAGP and FLAG. FIG. 10D is a graph of the results from a phagocytosis assay for uptake of pHrodo Red-labeled wildtype Ramos cells transfected with N-terminus FLAG-tagged wildtype and mutant SMAGP protein by wildtype mouse J774 macrophages, in the presence of anti-CD47 and anti-CD20 antibodies. Data were normalized to signals in GFP-FLAG control Ramos cells fed to J774 cells at the 12h timepoint (n=3 cell culture wells). FIG. 10E, left is a schematic of binding assays for FLAG-tagged wildtype and mutant SMAGP overexpressing Ramos cells with recombinant PILRa-Fc protein. FIG. 10E, right is flow cytometry results for human PILRa-Fc binding assay.

FIGS. 11A-11C show SMAGP-PILRa axis can be inhibited with anti-PILRa antibody and could be a potential target. FIG. 11A is images of pHrodo Red-labeled dCas9-VPR Ramos cells transfected with sgSafe or sgSMAGP incubated with Safe knockout J774 macrophages treated with anti-PILRa or isotype IgG control antibody at 9 h timepoint (error bar=100 μm). FIG. 11B is an exemplary working model of SMAGP-PILRa. FIG. 11C is a summary of hazard ratio (HR) comparing treatment outcome of SMAGP low expression patients to SMGAP high expression patients in various cancer types. HR<1.0 represents that low expression in SMAGP corresponds to a better treatment outcome. ACC: Adrenocortical carcinoma; PCPG: Pheochromocytoma and Paraganglioma; GBM: Glioblastoma multiforme; LGG: Brain Lower Grade Glioma; PRAD: Prostate adenocarcinoma; KIRC: Kidney renal clear cell carcinoma; SARC: Sarcoma; PAAD: Pancreatic adenocarcinoma; BRCA: Breast invasive carcinoma; LUAD: Lung adenocarcinoma; CHOL: Cholangiocarcinoma; READ: Rectum adenocarcinoma; UCEC: Uterine Corpus Endometrial Carcinoma; MESO: Mesothelioma; BLCA: Bladder Urothelial Carcinoma;THCA: Thyroid carcinoma; CESC: Cervical squamous cell carcinoma and endocervical adenocarcinoma; LIHC: Liver hepatocellular carcinoma; TGCT: Testicular Germ Cell Tumors; UVM: Uveal Melanoma; SKCM: Skin Cutaneous Melanoma; OV: Ovarian serous cystadenocarcinoma; UCS: Uterine Carcinosarcoma; LUSC: Lung squamous cell carcinoma; COAD: Colon adenocarcinoma; ESCA: Esophageal carcinoma; HNSC: Head and Neck squamous cell carcinoma; STAD: Stomach adenocarcinoma; DLBC: Lymphoid Neoplasm Diffuse Large B-cell Lymphoma; THYM: Thymoma; KICH: Kidney Chromophobe; KIRP: Kidney renal papillary cell carcinoma.

FIGS. 12A-12B show SMAGP is expressed across different tissues and can be overexpressed in the malignant cells. FIG. 12A is a comparison of SMAGP expression level between normal tissues and tumor tissues in different cancer types based on TCGA mRNA dataset. Wilcoxon signed-rank test was performed to evaluate the difference in SMAGP expression levels between normal tissues and tumor tissues. Cancer types that show statistically significant differences are marked by red asterisks (*** p<0.001). (BLCA: Bladder Urothelial Carcinoma; BRCA: Breast invasive carcinoma; COAD: Colon adenocarcinoma; ESCA: Esophageal carcinoma; NHSC: Head and Neck squamous cell carcinoma; KICH: Kidney Chromophobe; KIRC: Kidney renal clear cell carcinoma; KIRP: Kidney renal papillary cell carcinoma; LIHC: Liver hepatocellular carcinoma; LUAD: Lung adenocarcinoma; LUSC: Lung squamous cell carcinoma; PRAD: Prostate adenocarcinoma; STAD: Stomach adenocarcinoma; THCA: Thyroid carcinoma; UCEC: Uterine Corpus Endometrial Carcinoma) FIG. 12B is graphs of SMAGP expression level in different cell lines, grouped by cell line lineages.

FIGS. 13A-13B show PILRa can be knocked out in J774 mouse macrophage cell line or human primary blood monocyte cells-derived macrophages. FIG. 13A is a graph of ICE analysis results for PILRa knockout in Cas9 J774 macrophages. FIG. 13B is a graph of ICE analysis results for PILRa knockout in human primary blood monocyte cells-derived macrophages nucleofected with ribonucleoprotein targeting PILRa.

FIGS. 14A-14D show the combination of mutations in particular SMAGP glycosylation sites significantly inhibit SMAGP-mediated phagocytosis suppression and inhibit PILRa direct binding. FIG. 14A shows expression of N-terminus FLAG-tagged wildtype and mutant SMAGP in wildtype Ramos cells, as determined by western blot with antibodies against SMAGP and FLAG (negative results included). FIG. 14B shows surface expression of N-terminus FLAG-tagged wildtype and mutant SMAGP on wildtype Ramos cells, as determined by flow cytometry analysis with antibodies against FLAG. FIG. 14C is a graph of the results from a phagocytosis assay for uptake of pHrodo Red-labeled wildtype Ramos cells transfected with N-terminus FLAG-tagged wildtype and mutant SMAGP protein by wildtype mouse J774 macrophages, in the presence of anti-CD47 and anti-CD20 antibodies. Data were normalized to signals in GFP-FLAG control Ramos cells fed to J774 cells at the 12h timepoint (n=3 cell culture wells, negative results included). FIG. 14D is flow cytometry results for human PILRa-Fc binding assay (negative results included).

FIGS. 15A-15F show SMAGP expression level is related to clinical treatment outcome, and is not very highly expressed in most normal tissues. FIGS. 15A-15C are Kaplan-Meier plots for patients with glioblastoma multiforme (FIG. 15A, GBM, n=168), brain lower grade glioma (FIG. 15B, LGG, n=532), and adrenocortical carcinoma (FIG. 15C, ACC, n=79) with high or low SMAGP protein expression plotted from TCGA dataset. A 75% quantile split was used and curve separation was assessed by two-sided log-rank test (*P<0.0001). FIGS. 15D-15F are graphs of SMAGP (FIG. 15D), CD47 (FIG. 15E), and HER2 (ERBB2) (FIG. 15F) expression across different tissues from GETx Portal.

FIGS. 16A-16C show SMAGP expression level is associated with MYC expression in breast cancer and some other cancer types. FIG. 16A is SMAGP mRNA expression in different breast cancer integrative (IC) subtypes. FIG. 16B is SMAGP mRNA abundance is positively associated with MYC expression in five cancer types (LIHC: Liver hepatocellular carcinoma; PRAD: Prostate adenocarcinoma; BLCA: Bladder Urothelial Carcinoma; BRCA: Breast invasive carcinoma; KIRC: Kidney renal clear cell carcinoma). FIG. 16C shows SMAGP positively correlated with MYC transcriptional signature in METABRIC.

### DETAILED DESCRIPTION

Recently, the metastasis-linked gene SMAGP was identified through an unbiased genetic screening approach as an anti-phagocytic factor expressed at high levels in certain tumors and cancer cell lines. Herein, using genome-wide CRISPR screens in macrophages, the macrophage inhibitory receptor PILRA was identified as facilitating SMAGP-mediated suppression of phagocytosis. Deletion of PILRA in macrophages, or treatment of macrophages with anti-PILRA antibodies, abolished the effect of SMAGP on phagocytosis. These findings uncovered an inter-cellular signaling axis that controls phagocytosis and therapeutic targets to sensitize cancer cells to antibody-driven macrophage clearance.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

“Polynucleotide” or “oligonucleotide” or “nucleic acid,” as used herein, means at least two nucleotides covalently linked together. The polynucleotide may be DNA, both genomic and cDNA, RNA, or a hybrid, where the polynucleotide may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. Polynucleotides may be single- or double-stranded or may contain portions of both double stranded and single stranded sequence. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The proteins may be modified by the addition of sugars, lipids or other moieties not included in the amino acid chain. The terms “polypeptide” and “protein” are used interchangeably herein.

“Antibody” and “antibodies” as used herein refers to monoclonal antibodies, monospecific antibodies (e.g., which can either be monoclonal, or may also be produced by other means than producing them from a common germ cell), multi-specific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25 (11): 1290-1297 (2007) and PCT International Application WO 2001/058956, the contents of each of which are herein incorporated by reference), or domain antibodies (dAbs) (e.g., such as described in Holt et al., Trends in Biotechnology 21:484-490 (2014)), and including single domain antibodies sdAbs that are naturally occurring, e.g., as in cartilaginous fishes and camelid, or which are synthetic, e.g., nanobodies, VHH, or other domain structure), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass. For simplicity sake, an antibody against an analyte is frequently referred to herein as being either an “anti-analyte antibody” or merely an “analyte antibody”.

“Antibody fragment” as used herein refers to a portion of an intact antibody comprising the antigen-binding site or variable region. The portion does not include the constant heavy chain domains (i.e., CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

As used herein, “treat.” “treating,” and the like mean a slowing, stopping, or reversing of progression of a disease or disorder when provided a composition described herein to an appropriate control subject. The term also means a reversing of the progression of such a disease or disorder to a point of eliminating or greatly reducing the cell proliferation. As such. “treating” means an application or administration of the methods described herein to a subject, where the subject has a disease or a symptom of a disease, where the purpose is to cure, heal, alleviate, relieve, alter, remedy, ameliorate, improve, or affect the disease or symptoms of the disease.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice, and guinea pigs, and the like. Examples of non-mammals include, but are not limited to, birds, fish, and the like. In one embodiment of the methods and compositions provided herein, the mammal is a human.

The term “contacting” as used herein refers to bring or put in contact, to be in or come into contact. The term “contact” as used herein refers to a state or condition of touching or of immediate or local proximity. Contacting inhibitors of the disclosed methods to a target destination, such as, but not limited to, an organ, tissue, cell, or tumor, may occur by any means of administration known to the skilled artisan.

As used herein, the terms “providing,” “administering,” and “introducing” are used interchangeably herein and refer to the placement into a subject by a method or route which results in at least partial localization to a desired site. The inhibitors of the disclosed methods can be administered by any appropriate route which results in delivery to a desired location in the subject.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Enhancing Phagocytosis

Phagocytosis is a basic process for nutrition in unicellular organisms and is found in almost all cell types of multicellular organisms. A specialized group of cells (e.g., macrophages, neutrophils, monocytes, dendritic cells, osteoclasts) accomplish phagocytosis with high efficiency and are primarily responsible for the removal of microorganisms and presentation of antigens to lymphocytes as part of the adaptive immune response. Other cell types can also participate in more general uses for phagocytosis including eliminating dead cells and maintaining homeostasis. The present disclosure provides methods for enhancing phagocytosis of a target cell.

In some embodiments, the methods comprise contacting an immune cell with an inhibitor of paired immunoglobulin-like type 2 receptor-alpha (PILRA). In some embodiments, the immune cell comprises an innate immune cell. Innate immune cells include, without limitation, basophils, dendritic cells, eosinophils, Langerhans cells, mast cells, monocytes and macrophages, neutrophils, and NK cells. In some embodiments, the innate immune cell is a monocyte, a neutrophil, or a macrophage.

The methods are not limited by type or nature of the target cell. The target cell may be any cell type. The target cell may be a cell in vitro, either from a cell line or cells obtained from a subject (ex vivo). The target cell may be in vivo. Thus, in some embodiments, contacting an immune cell comprises administration to a subject in need thereof.

In some embodiments, the target cell overexpresses small cell adhesion glycoprotein (SMAGP).

In some embodiments, the target cell is a cancer cell. The methods are not limited by type or source of cancer cell. In some embodiments, the cancer cell comprises lymphoma, cervical cancer, lung cancer, colorectal cancer, ovarian cancer, breast cancer and/or leukemia.

Accordingly, the methods may be used to treat a disease or disorder. In some embodiments, the disease or disorder is characterized by suppression of phagocytosis. In some embodiments, the disease or disorder is characterized by the over-proliferation of a diseased cell. The disease or disorder may comprise an autoimmune disorder, cancer, or atherosclerosis.

In some embodiments, the disease or disorder is cancer. In some embodiments, the cancer comprises a solid tumor. In some embodiments, the cancer is metastatic cancer. In some embodiments, the disclosed methods result in suppression of elimination of metastasis. In some embodiments, the disclosed methods result in decreased tumor growth. In some embodiments, the disclosed methods prevent tumor recurrence. In instances when the disease or disorder is cancer, the target cell may be a cancer cell.

The disclosed methods may be useful to treat a wide variety of cancers including carcinoma, sarcoma, lymphoma, leukemia, melanoma, mesothelioma, multiple myeloma, or seminoma. The cancer may be a cancer of the bladder, blood, bone, brain, breast, cervix, colon/rectum, endometrium, head and neck, kidney, liver, lung, lymph nodes, muscle tissue, ovary, pancreas, prostate, skin, spleen, stomach, testicle, thyroid, or uterus. In select embodiments, the cancer comprises lymphoma, cervical cancer, lung cancer, colorectal cancer, ovarian cancer, breast cancer and/or leukemia.

In some embodiments, the disease or disorder is an autoimmune disorder. Autoimmune diseases and disorders refer to conditions in a subject characterized by cellular, tissue and/or organ injury caused by an immunologic reaction of the subject to its own cells, tissues and/or organs. Autoimmune diseases and disorders that may be treated by the methods of the present invention include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), irritable bowel disease (IBD). IgA neuropathy, juvenile arthritis, lichen planus, lupus erythematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychondritis, polyglandular syndromes, polymyalgia rheumatics, polymyositis and dermatomyositis, primary agammaglobulinemia, primary biliary cirrhosis, psoriasis, psoriatic arthritis. Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, takayasu arteritis, temporal arteritis/giant cell arteritis, ulcerative colitis, uveitis, vasculitides such as dermatitis herpetiformis vasculitis, vitiligo, and Wegener's granulomatosis. In select embodiments, the disease or disorder comprises rheumatoid arthritis or multiple sclerosis.

In some embodiments, the disease or disorder is atherosclerosis. Atherosclerosis comprises any disease or disorder characterized by the deposition of fats, cholesterol, and other substances in and on the walls of an artery causing the arteries to narrow thereby blocking blood flow or leading to a blood clot. Atherosclerosis and atherosclerotic associated diseases can affect arteries anywhere in your body and include but are not limited to coronary heart disease, carotid artery disease, chronic kidney disease and peripheral arterial disease.

Thus, in some embodiments, the methods comprise administering to a subject in need thereof an inhibitor of paired immunoglobulin-like type 2 receptor-alpha (PILRA).

In some embodiments, the methods further comprise administering to the subject or contacting the target cell with an inhibitor of at least one anti-phagocytic factor. Anti-phagocytic factors include those that prevent or negatively control phagocytosis. Anti-phagocytic factors and methods of identifying anti-phagocytic factors are known. See for example, International Patent Publication No. WO 2022/076446, incorporated herein by reference in its entirety. In some embodiments, the anti-phagocytic factor is small cell adhesion glycoprotein (SMAGP).

Inhibitors (e.g., inhibitors of PILRA and or SMAGP) may be any substance (e.g., nucleic acid, proteins, polysaccharides, nucleotides, amino acids, monosaccharides or simple sugars, small molecules) which modulates (e.g., inhibits) the transcription, translation, or action of the referenced target in a specific manner. In some embodiments, the inhibitors include nucleic acid based substances and systems which modulate transcription or translation, including but not limited to, small interfering RNA or CRISPR knockout systems. In some embodiments, the inhibitors include substances and systems which modulate the action of the gene product, including but not limited to, antibodies and small molecule inhibitors.

For example, PILRA inhibitors include, but are not limited to, PILRA antibodies 2175D, 36H2, H2, and those antibodies disclosed in U.S. Patent Publication No. 2019/0211098, incorporated herein by reference, PILRA siRNAs and shRNA (e.g., SIRGT77617WQ-2F and SIRGT96479WQ-2Ome, Creative Biolabs). In some embodiments, the PILRA inhibitor is not an antibody.

SMAGP inhibitors include but are not limited to those antibodies disclosed in U.S. Patent Publication No. 2005/0032101, incorporated herein by reference, and siRNAs as disclosed in Jia et al., Onco Targets Ther. 2018; 11:6925-6935, incorporated herein by reference.

The method may comprise administering to a subject, in vivo an effective amount of the described inhibitors. In some embodiments, the described inhibitors are delivered to a tissue of interest by, for example, an intramuscular, intravenous, transdermal, intranasal, oral, mucosal, or other delivery methods.

When utilized as a method of treatment, the effective amount may depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

The described inhibitors may be administered as a composition which further comprises a pharmaceutically acceptable carrier. The phrase “pharmaceutically acceptable,” as used in connection with compositions and/or cells of the present disclosure, refers to molecular entities and other ingredients of such compositions that are physiologically tolerable and do not typically produce untoward reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g., Remington: The Science and Practice of Pharmacy 20th Ed. (2000) Lippincott Williams and Wilkins, Ed. K. E. Hoover.

Thus, the present disclosure provides compositions comprising an inhibitor of paired immunoglobulin-like type 2 receptor-alpha (PILRA) and an inhibitor of at least one anti-phagocytic factor, as described elsewhere herein. In some embodiments, the at least one anti-phagocytic factor comprises small cell adhesion glycoprotein (SMAGP).

In some embodiments, the compositions further comprise at least one or both of a tumor antigen (TA)-targeting antibody and a CD47 blocking antibody, as described elsewhere herein. In some embodiments, the TA-targeting antibody is a CD20 blocking antibody. In some embodiments, the CD47 blocking antibody comprises an anti-CD47 antibody, an anti-SIRPalpha antibody, or any combination thereof.

A wide range of second therapies may be used with the disclosed methods. The second therapy may be administration of a therapeutic agent or may be a second therapy not connected to administration of another agent. Such second therapies include, but are not limited to, surgery, immunotherapy, radiotherapy, a chemotherapeutic or anti-cancer agent, a statin or other cholesterol controlling medication, a blood thinner, and blood pressure medications.

As used herein, the term “chemotherapeutic” or “anti-cancer agent” includes any small molecule or other drug used in cancer treatment or prevention. Chemotherapeutics include, but are not limited to, cyclophosphamide, methotrexate, 5-fluorouracil, doxorubicin, docetaxel, daunorubicin, bleomycin, vinblastine, dacarbazine, cisplatin, paclitaxel, raloxifene hydrochloride, tamoxifen citrate, abemacicilib, afinitor, alpelisib, anastrozole, pamidronate, anastrozole, exemestane, capecitabine, epirubicin hydrochloride, eribulin mesylate, toremifene, fulvestrant, letrozole, gemcitabine, goserelin, ixabepilone, emtansine, lapatinib, olaparib, megestrol, neratinib, palbociclib, ribociclib, talazoparib, thiotepa, toremifene, methotrexate, and tucatinib.

The second therapy (e.g., an immunotherapy) may be administered at the same time as the disclosed methods, either in the same composition or in a separate composition administered at substantially the same time. In some embodiments, the second therapy may precede or follow the disclosed methods by time intervals ranging from hours to months.

In some embodiments, the second therapy includes immunotherapy. In some embodiments, the second therapy does not include immunotherapy. Immunotherapies include chimeric antigen receptor (CAR) T-cell or T-cell transfer therapies, cytokine therapy, immunomodulators, cancer vaccines, or administration of antibodies (e.g., monoclonal antibodies).

In some embodiments, the immunotherapy comprises administration of antibodies. The antibodies may target antigens either specifically expressed by tumor cells or antigens shared with normal cell. In some embodiments, the immunotherapy may comprise an antibody targeting, for example, CD20, CD33, CD52, CD30, HER (also referred to as erbB or EGFR), VEGF, CTLA-4 (also referred to as CD152), epithelial cell adhesion molecule (EpCAM, also referred to as CD326), and PD-1/PD-L1. Suitable antibodies include, but are not limited to, rituximab, blinatumomab, trastuzumab, gemtuzumab, alemtuzumab, ibritumomab, tositumomab, bevacizumab, cetuximab, panitumumab, ofatumumab, ipilimumab, brentuximab, pertuzumab, and the like). In some embodiments, the immunotherapy does not comprise an antibody targeting any one or more of CD20, CD33, CD52, CD30, HER (also referred to as erbB or EGFR), VEGF, CTLA-4 (also referred to as CD152), epithelial cell adhesion molecule (EpCAM, also referred to as CD326), PD-1, and PD-L1. In some embodiments, the immunotherapy does not comprise an antibody targeting the PD-1/PD-L1 interaction.

In some embodiments, the immunotherapy comprises contacting the cell with at least one or both of a tumor antigen (TA)-targeting antibody and a CD47 blocking antibody. Any known TA-targeting or CD47 blocking antibody or blocking agent may be compatible with the disclosed methods, including, for example, anti-EGFR agents (e.g., cetuximab), anti-CD30 agents (e.g., brentuximab), an anti-CD47 antibody, an anti-SIRPalpha antibody, soluble SIRPa fragments, CD20 antibodies (e.g., rituximab, obinutuzumab, ofatumumab), and the like. In some embodiments the TA-targeting antibody comprises rituximab, cetuximab, brentuximab, or a combination thereof. In some embodiments, the CD47 blocking antibody comprises an anti-CD47 antibody, an anti-SIRPalpha antibody, or any combination thereof.

The antibodies may also be linked to a chemotherapeutic agent. Thus, in some embodiments, the antibody is an antibody-drug conjugate.

### 3. Examples

### Materials and Methods

Cell Culture Ramos and RKO cells were maintained in T-75 flasks or 10-cm plates in RPMI-1640 media supplemented with 2 mM glutamine, 100 U ml-1 penicillin, 100 μg ml-1 streptomycin and 10% fetal bovine serum (FBS). J774 cells were cultured in 10-cm plates in DMEM media supplemented with 2 mM glutamine, 100 U ml-1 penicillin, 100 μg ml-1 streptomycin and 10% heat-inactivated FBS, and were passaged by exchanging media when cells reached ˜90% confluency, incubating for 24 h, and scraping. All cells were cultured in a humidified 37° C. incubator set at 5% CO2. Cells were passaged two to three times weekly. To generate frozen aliquots, cells were pelleted by centrifugation (350 g, 5 min, room temperature), resuspended in either 90% FCS with 10% dimethylsulfoxide (DMSO) or manufactured frozen media Bambanker, and frozen in cell-freezing containers at −80° C. overnight before transfer to liquid nitrogen for long-term storage.

Construction of Cell Lines For generating individual knockout and overexpression lines, RKO and J774 cells stably expressing Cas9, and Ramos cells stably expressing dCas9-VPR were infected with lentiviral constructs expressing a given sgRNA along with puromycin resistance and GFP marker. Cells were incubated in virus for 1-3 days before they were allowed to recover for 1 day, and were then selected with 1 μg ml-1 puromycin for 3 days or until >95% of cells were GFP+.

Screen in RKO cells For the CRISPR knockout screen in RKO cells, a previously described 730-gene CRISPR deletion library (Kamber et al. Nature 597, 549-554 (2021), incorporated herein by reference in its entirety) enriched for phagocytosis regulators, with 10 sgRNAs per gene, was introduced into Cas9-expressing RKO cells at a multiplicity of infection (MOI) of ˜0.2. Cells were selected with puromycin (1 μg ml-1) for 3 d, then allowed to recover and expand in puromycin-free media. In two replicates, the RKO knockout pool was then either exposed to LPS-stimulated (100 ng ml-1, 24 h pre-treatment) J774 macrophages in the presence of anti-CD47 (clone B6.H12, BioXCell) (2.5 μg ml-1), or left untreated. During the screens, the libraries were propagated at ˜2000× coverage of the library. RKO cells were then allowed to recover, and then subjected to another round of treatment. At the end of the screen, 50 million cells were recovered from each condition and pelleted by centrifugation. Genomic DNA of each condition was extracted using a DNA Blood Maxi kit (Qiagen, Cat. #51194) and preparation of sequencing libraries was conducted as described previously (Kamber et al. 2021). Hits were identified using CasTLE (Morgens et al. Nature Biotechnology 34 (6): 634-36 (2016), incorporated herein by reference in its entirety).

Screen in J774 macrophages For the CRISPR knockout screen in J774 cells to identify mediators of phagocytosis suppression by cancer-cell SMAGP, a 2,208-gene phagocytosis-regulator enriched library of sgRNAs was integrated into J774 Cas9 cells, as previously described. The macrophage knockout library was plated in 15 cm tissue culture dishes (5 million cells per plate), cultured for 24 h, and then treated for 24 h with 100 ng ml-1 LPS. Macrophages were then co-incubated with a mixture of calcein-labeled GFP-FLAG-expressing Ramos cells and CellTrace Far-red-labeled SMAGP-FLAG expressing Ramos cells for 24 h in the presence of anti-CD20 and anti-CD47. The macrophages were washed with PBS to remove unphagocytosed Ramos cells, and then were lifted by scraping, washed two additional times in PBS, and then separated into four populations on an FACSAria (BD) cell sorter. In total, ˜10 million cells were collected for each replicate across the 4 gated populations. Extraction of genomic DNA, amplification of sgRNA-encoding loci, next-generation sequencing, and hit identification with CasTLE were performed as described previously (Kamber et al. 2021).

Phagocytosis Assays For use in time-lapse microscopy phagocytosis assays, J774 cells were plated at a density of 50,000 cells per well in 24-well tissue culture plates 48 h before the start of the assay. At 24 h after plating, medium was aspirated and replaced with 0.5 ml medium containing 100 ng ml-1 LPS (Sigma). At the day of the assay, Ramos or RKO cells that were transduced with sgRNAs were counted, washed once with PBS, and incubated in PBS containing 100 nM pHrodo-Red succinimidyl ester (Thermo Fisher) at a concentration of 1 million cells ml-1 for 15 min in 37° C. tissue culture incubator in the dark. pHrodo-stained target cells were pelleted, washed once in complete DMEM media (DMEM supplemented with 2 mM glutamine, 100 U ml-1 penicillin, 100 μg ml-1 streptomycin and 10% heat-inactivated FBS), and incubated in complete DMEM media containing anti-CD20 (500 ng ml-1) and anti-CD47 (10 μg ml-1) at a concentration of 1 million cells ml-1 for 10 min at room temperature. For the pre-treatment of macrophages with anti-PILRa antibody, medium was aspirated and replaced with 100 μl media containing anti-PILRa antibody (4 μg ml-1). J774 macrophages were then incubated in the anti-PILRa containing media for 10 min at room temperature. 250,000 Ramos cells or 50,000 RKO cells were then added to each well and allowed to settle. Plates were transferred to an incubator and 9 images were taken per well for every hour using an Incucyte (Sartorius). Total integrated red intensity per well was calculated and used to measure phagocytosis. Assays with PBMCs were conducted as described previously using cells from Leukocyte Reduction Chambers (provided by the Stanford Blood Center from healthy donors) (Kamber et al. 2021).

Isolation of human peripheral blood mononuclear cell derived macrophages Leukocyte reduction system (LRS) chambers from anonymous donors were obtained from the Stanford Blood Center. Cells were separated using Ficoll-Paque gradient centrifugation. Monocytes were then isolated by allowing the cells to grow and adhere to tissue culture treated plates. The cells were differentiated into macrophages by culturing in RPMI-1640 containing 20% heat-inactivated FBS and 20 ng ml-1 human M-CSF (Peprotech) for 5 days, then lifted with Accutase and scraping. The harvested cells were counted and seeded to 24-well plates in 200,000 cells per well, and allowed to adhere overnight. Macrophages were then treated for 24 h with 100 ng ml-1 LPS (Sigma) before used in phagocytosis assays.

|0079| Human primary macrophage genetic alteration sgRNA molecules targeting the PILRa locus and off-target Safe loci were purchased as modified, hybridized RNA molecules from Synthego. The sgRNA sequences were as follows: sgRNA PILRa, 5′-CCGTCCCCTGGAGAAGAACA-3′ (SEQ ID NO: 1); and Safe sgRNA, 5′-AGGCCCCCUCCCAGGCGUAG-3′ (SEQ ID NO: 2). SpCas9 protein was purchased from IDT. Cas9 ribonucleoprotein was formed by intubation of Cas9 protein with sgRNAs at a molar ratio of 1:2.5 at 37° C. for 30 min. At Day 7 post isolation, the human primary macrophages were electroporated with pre-formed ribonucleoprotein using following the protocol provided by the P3 Primary Cell Nucleofection Kit (Lonza, V4XP-3024) and the Lonza Nucleofector 4D. Nucleofected macrophages were allowed to recover for a short while in culture media before they were counted and seeded in 24 well plates at a density of 200k cells/well. The seeded as well as extra nucleofected macrophages were cultured for an additional 48 h, after which phagocytosis assays were performed. Genomic DNA was also collected and TIDE analysis was performed to characterize the INDEL frequencies.

Western blot IM cells were collected, washed once in 1 mL PBS, and lysed with 150 μL lysis buffer (50 mM Tris-HCl pH 7.5, 150 mM NaCl, 1 mM EDTA, 1% Triton X-100, 1× complete protease inhibitor cocktail (Roche)) were heated in SDS loading buffer and separated by SDS-PAGE by NuPAGE 4-12% Bis-Tris Protein Gels (NP0322BOX, ThermoFisher), transferred to nitrocellulose, blotted and imaged using an Odyssey CLx (LI-COR Biosciences) or Supersignal West Femto Maximum Sensitivity Substrate with a Chemidoc System (Bio-Rad). Cell pellets were resuspended directly in the SDS loading buffer, sonicated, and separated by SDS-PAGE. The following antibodies were used: goat polyclonal anti-SMAGP (AF3959, R&D Systems, 1:1,000 dilution), mouse monoclonal anti-Flag (clone M2, F1804, Sigma, 1:2,000 dilution), and rabbit polyclonal anti-β-actin (ab8227, Abcam, 1:2,000 dilution).

### Example 1

### Identification of SMAGP as a Major Regulator of Phagocytosis Resistance in RKO Colon Cancer Cells

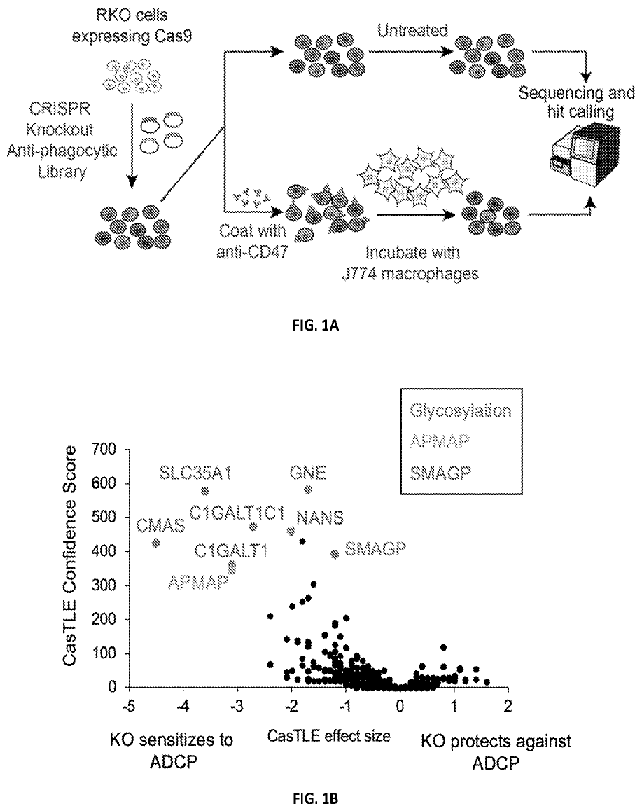

To prioritize anti-phagocytic factors that mediate cancer cell resistance against phagocytosis, a CRISPR screen was conducted in the phagocytosis-resistant colon cancer cell line, RKO (FIG. 1A). A 730-gene library corresponding to candidate anti-phagocytic factors identified in previous genome-wide CRISPR knockout and CRISPRa screens was used. Following two rounds of treatment of the RKO knockout library with macrophages and anti-CD47, the sgRNAs in the treated and untreated populations were sequenced to identify genes that are required for RKO resistance to phagocytosis. The top hits in this screen included many of the genes previously identified in CRISPR knockout screens as critical for lymphoma resistance to phagocytosis, including APMAP and several genes involved in sialic acid synthesis (FIG. 1B). Additionally, SMAGP, a metastasis-linked cell surface glycoprotein that had previously been identified in a CRISPRa screen in lymphoma cells as a gene whose overexpression is sufficient to protect cancer cells against phagocytosis, was identified as one of the top hits. This finding was consistent with a previous study showing that deletion of SMAGP in RKO cells rendered them sensitive to ADCP, and additionally demonstrated that SMAGP is a major regulator of ADCP sensitivity in this cell line.

### Example 2

### SMAGP Suppresses Cancer Cell Phagocytosis by Primary Human Macrophages

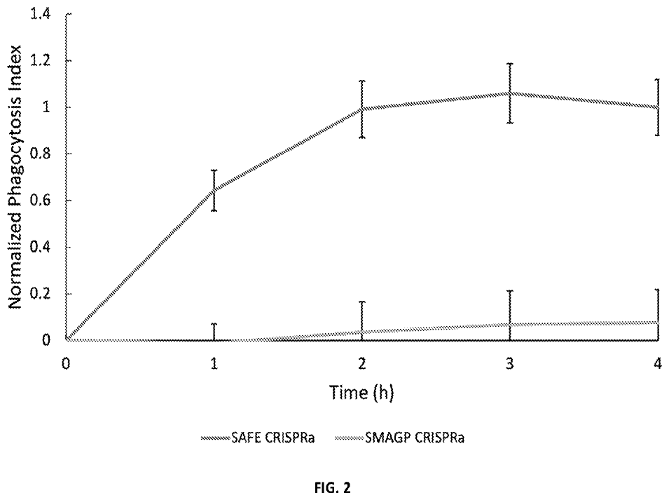

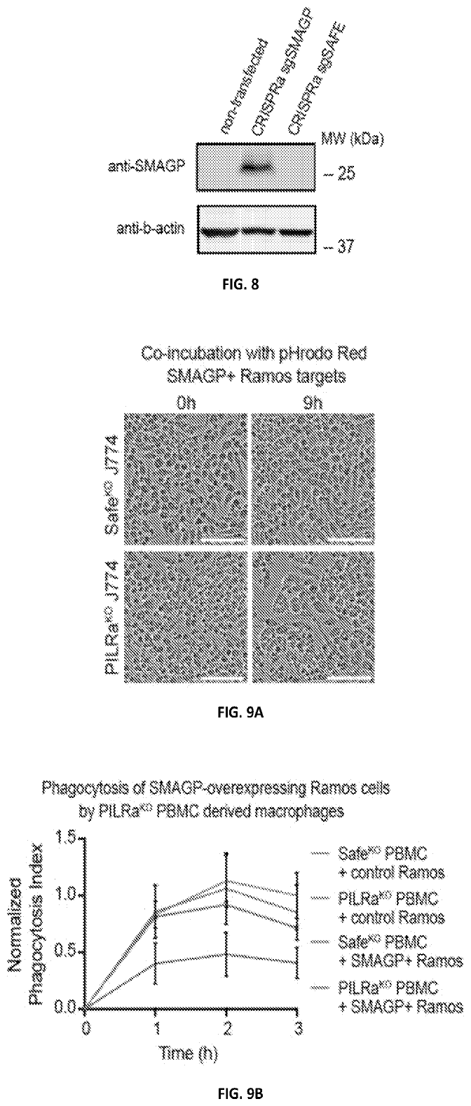

To validate that overexpression of SMAGP is sufficient to protect cancer cells against phagocytosis by primary human macrophages, Ramos lymphoma cells lines that overexpress either FLAG-tagged SMAGP or GFP were generated, and the lymphoma cells were incubated with LPS-activated human blood-derived primary macrophages in the presence of anti-CD47 and anti-CD20 antibodies to stimulate phagocytosis. Western blot confirmed that SMAGP was overexpressed in CRISPRa sgSMAGP Ramos cells (FIG. 8). PHrodo red-labeled SMAGP+ and SMAGP-Ramos cells were co-incubated with LPS-activated human blood-derived primary macrophages, with the presence of anti-CD47 and anti-CD20 antibodies to stimulate phagocytosis. Phagocytosis events represented by total red signal confirmed that overexpression of SMAGP in lymphoma cells was sufficient to protect cancer cells against phagocytosis of human macrophages, confirming that findings with mouse macrophages are recapitulated with primary human macrophages (FIG. 2).

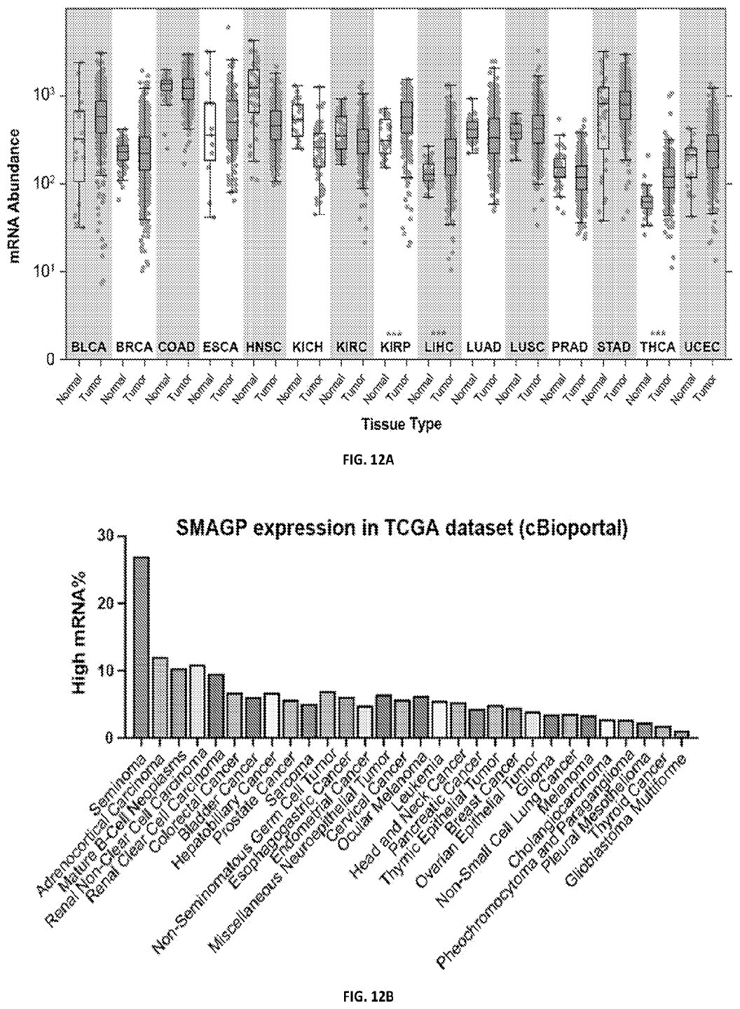

Analysis of SMAGP gene expression in primary cancer data from The Cancer Genome Atlas (TCGA) indicated that SMAGP is overexpressed in multiple cancer types, with five of them showing statistically significant higher expression in tumor tissues compared to normal tissue (FIG. 12A). Previous studies found that SMAGP serves as a marker in cervical cancer, and as well has higher expression in primary and metastasized colon, lung, breast cancers, and pancreatic adenocarcinoma. The analysis results from the TCGA dataset partially matched what has been reported (SMAGP is highly expressed in pancreatic malignancy), and identified additional tissues in which higher SMAGP expression is associated with cancer (liver, kidney, thyroid).

### Example 3

### Identification of PILRA as a Candidate Regulator of SMAGP-Mediated Resistance to Phagocytosis

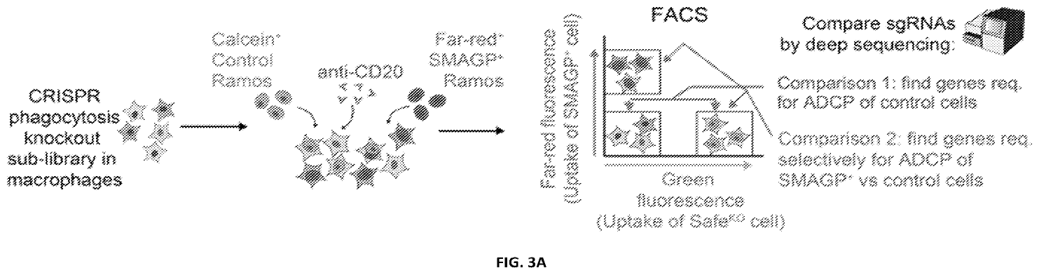

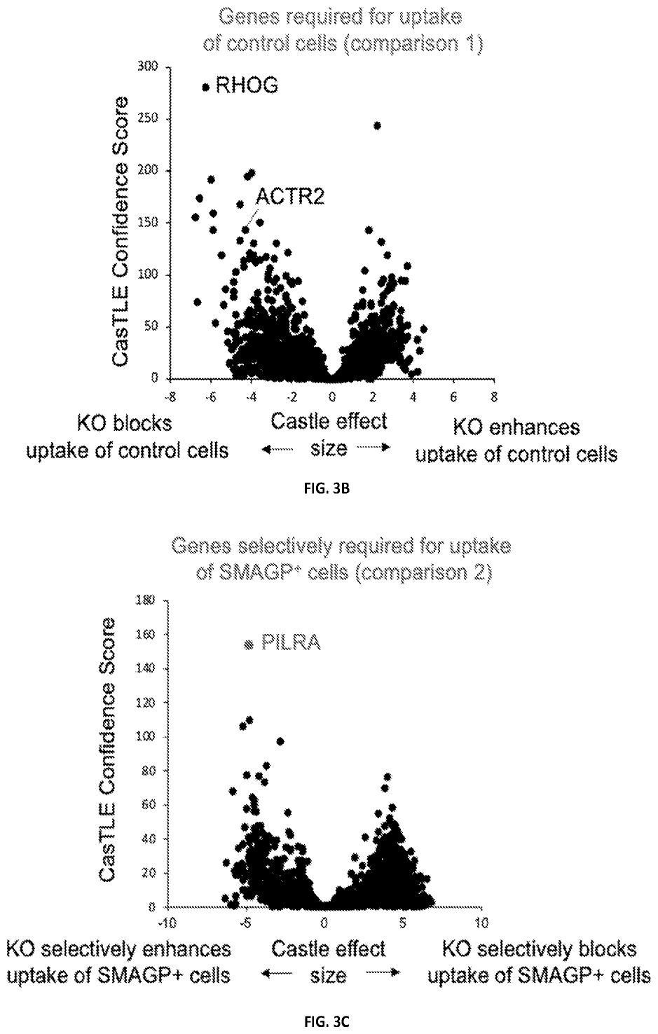

To identify a putative macrophage receptor, a recently-developed inter-cellular CRISPR screening approach was used. Specifically, a knockout library was integrated into macrophages that contained 2,208 genes, enriched for known regulators of phagocytosis as well as receptor protein families expressed in immune cells. This macrophage knockout pool was then incubated with differentially-fluorescently labeled control and SMAGP-overexpressing cells in the presence of anti-CD20 and anti-CD47 antibodies, to drive uptake of both cell types. The macrophage knockout population was subsequently separated by FACS into populations corresponding to macrophages that had phagocytosed a control lymphoma cell or a SMAGP-overexpressing lymphoma cell. By sequencing the sgRNAs in each population and comparing their distributions using CasTLE, genes that specifically regulated the phagocytosis of SMAGP-overexpressing lymphoma cells were identified (FIG. 3A).

Strikingly, this screen revealed the immune inhibitory receptor PILRA as the top hit (FIGS. 3B and 3C). Inspection of the sequencing data indicated that macrophages expressing sgRNAs targeting PILRA were enriched in the population of macrophages that had phagocytosed a SMAGP-overexpressing lymphoma cell.

### Example 4

### PILRA is Required for Suppression of Phagocytosis by Overexpressed SMAGP

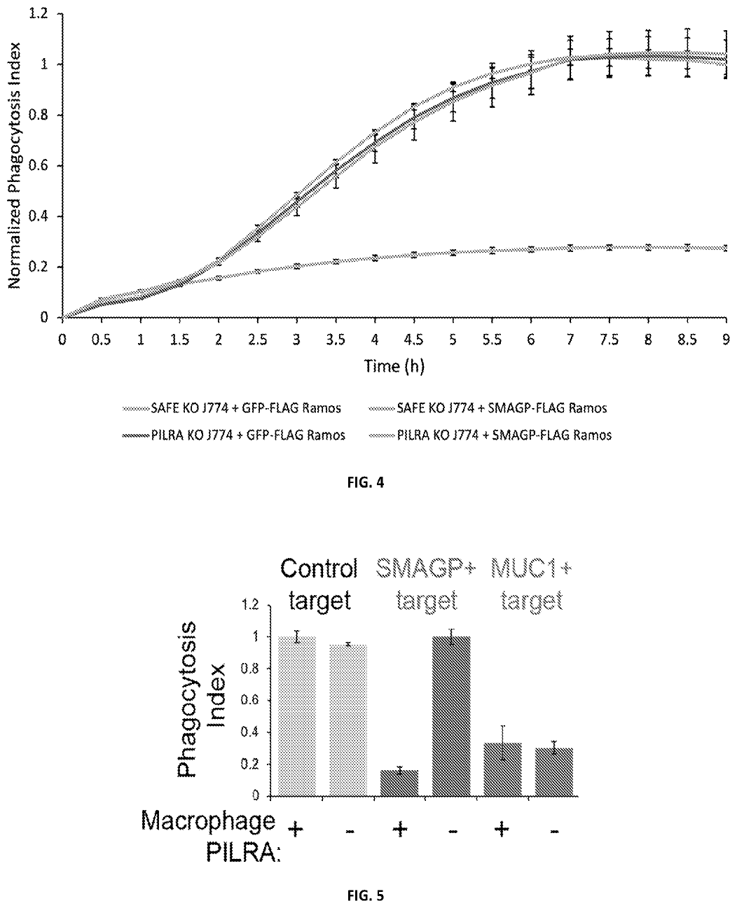

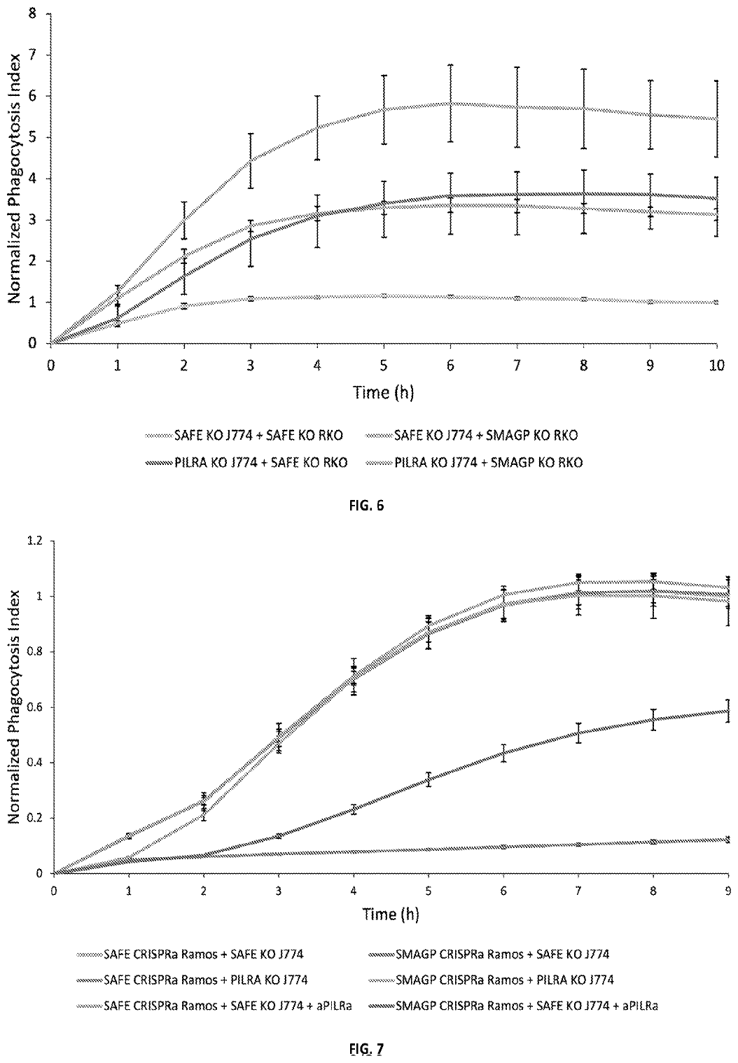

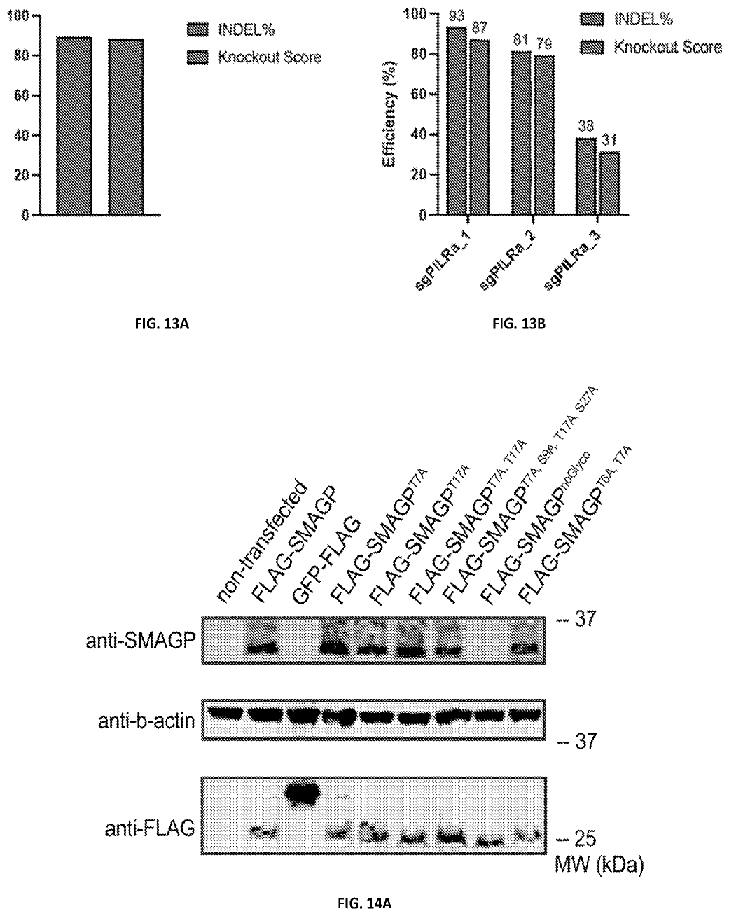

To test whether the suppressive effect of SMAGP on phagocytosis is dependent on macrophage PILRA, PILRA-knockout macrophages were generated (FIG. 13A) and co-incubated with SMAGP- or GFP-overexpressing Ramos lymphoma cells in the presence of anti-CD47 and anti-CD20 antibodies. Consistent with the phenotype measured in the screen, the ability of SMAGP-overexpressing lymphoma cells to suppress phagocytosis was abolished in the presence of PILRA-knockout macrophages (FIGS. 4 and 9A). To further investigate the necessity of PILRa in the function of SMAGP to protect cancer cells against phagocytosis, a protocol was adapted to genetically modify the expression of PILRa in primary derived human macrophages through the CRISPR-Cas9 ribonucleoprotein system. 93% of indel was confirmed through TIDE analysis 48 h following nucleofection with sgRNA targeting the PILRa locus, relative to its expression in cells nucleofected with the control Safe sgRNA (FIG. 13B). In the presence of anti-CD47 and anti-CD20 antibodies, the ability of SMAGP+Ramos cells to suppress phagocytosis decreased when incubating with PILRa-knockout macrophages (FIG. 9B). Additionally, loss of PILRA in macrophages sensitized RKO cancer cells, which endogenously express high levels of SMAGP, to phagocytosis (FIG. 6). The results in both mouse J774 and human primary macrophages indicated that PILRa facilitates phagocytosis suppression by SMAGP overexpression.

PILRa knockout and control J774 macrophages were also co-incubated with Ramos cells overexpressing MUC1, another sialylated cell surface protein that was identified as an anti-phagocytic factor in previously reported genome-wide screens. It was found that whereas PILRA deletion blocked the effect of SMAGP on phagocytosis, it had no effect on the ability of MUC1, another sialylated cell surface protein previously identified as an anti-phagocytic factor, to suppress phagocytosis (FIG. 5). Collectively, these findings indicate that the macrophage-expressed immune inhibitory receptor PILRA facilitates the ability of cancer-cell SMAGP to protect cancer cells from macrophage-mediated phagocytosis.

### Example 5

### Polyclonal Anti-PILRA Antibodies Inhibit SMAGP-Mediated Phagocytosis Suppression

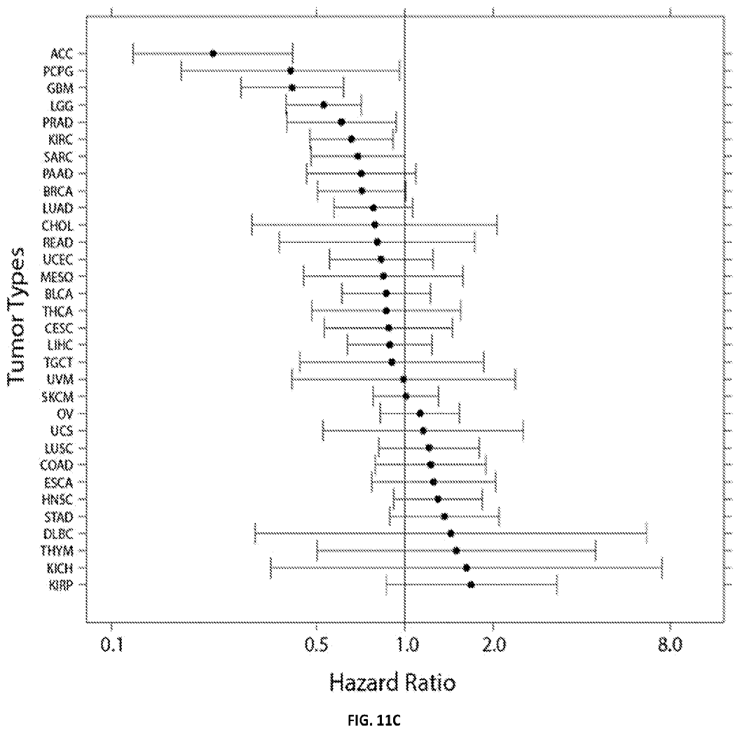

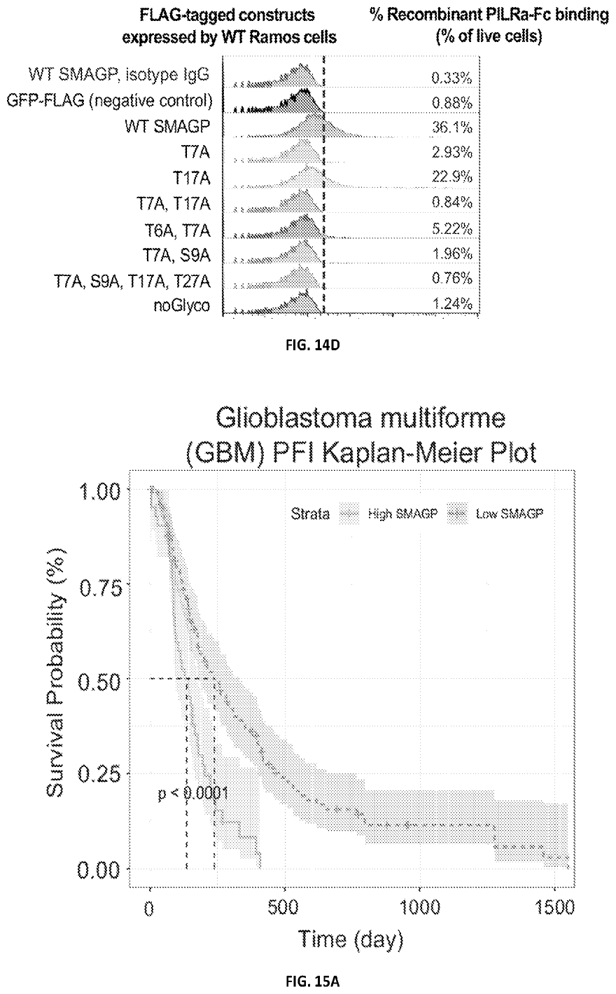

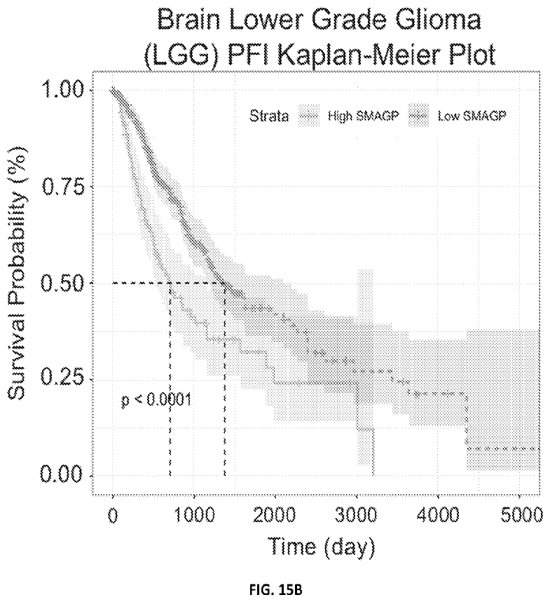

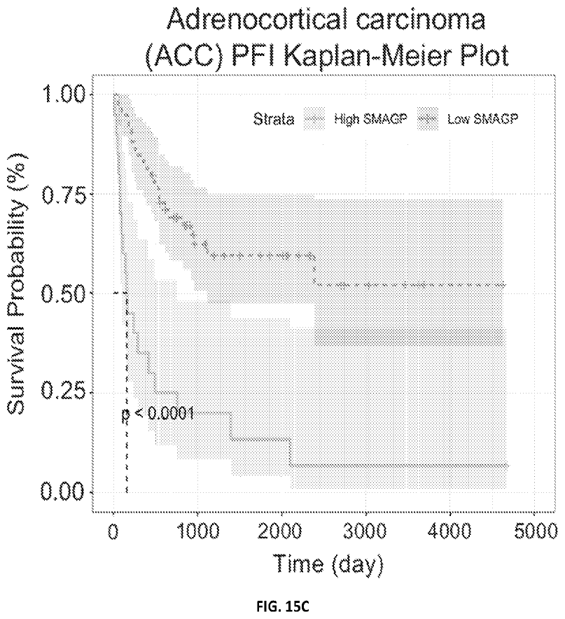

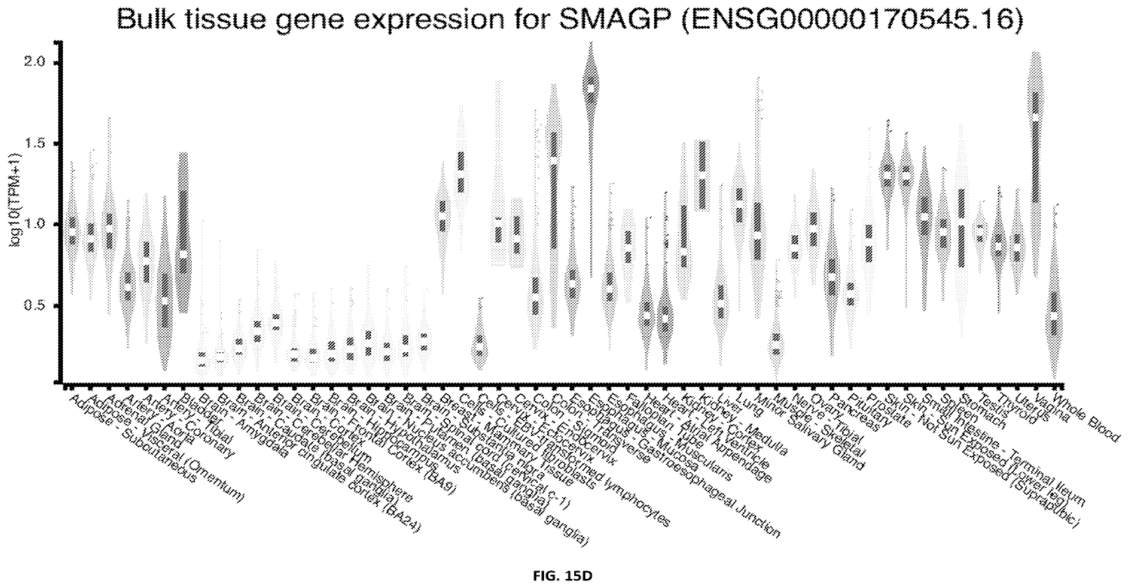

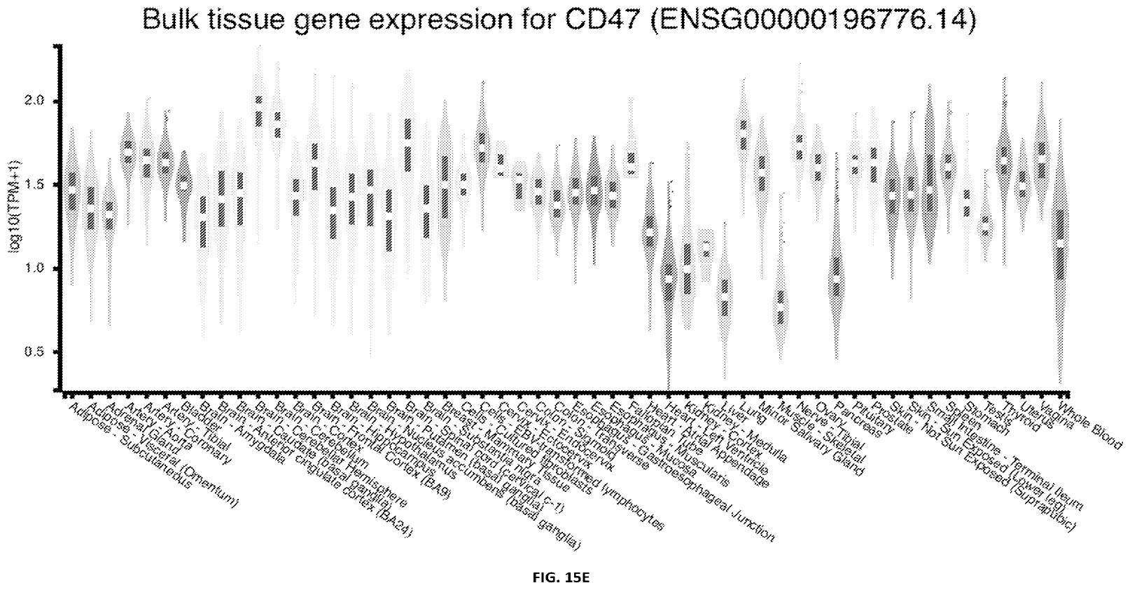

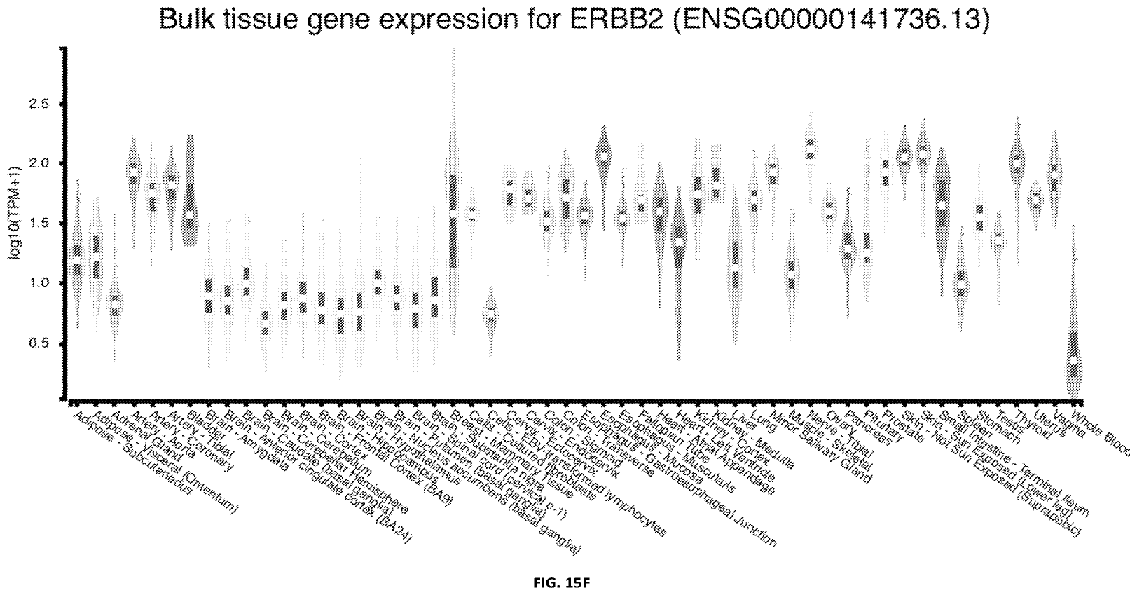

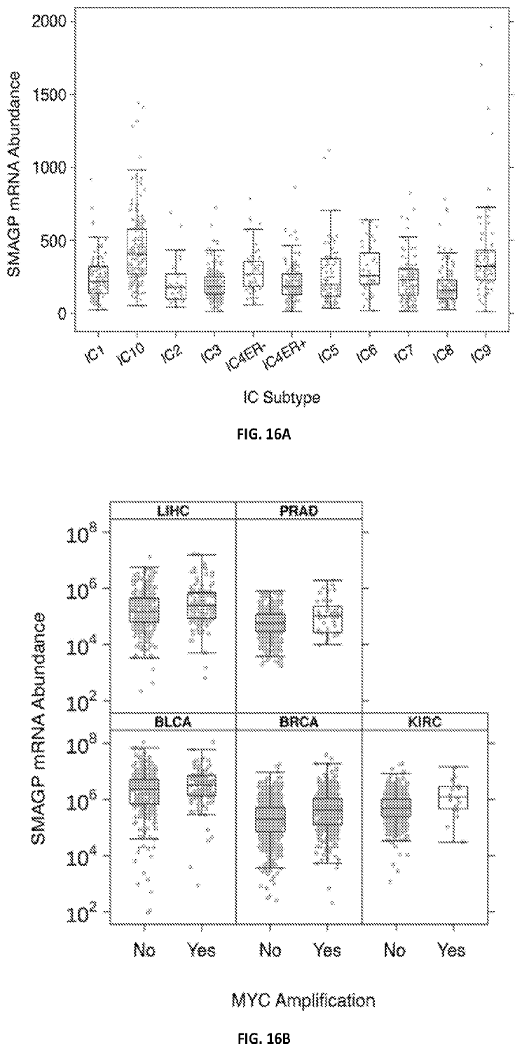

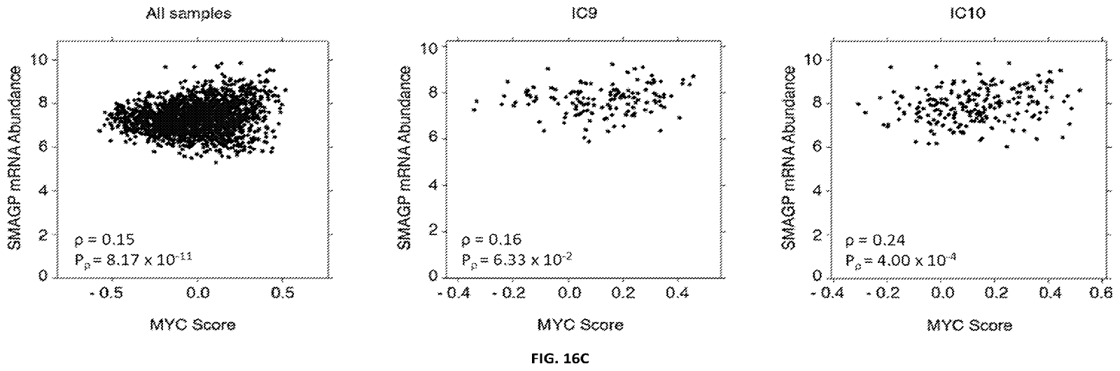

To determine whether acute blockade of PILRA function with neutralizing antibodies decreases the ability of SMAGP to suppress phagocytosis, PILRA-knockout macrophages were co-incubated with SMAGP- or GFP-overexpressing Ramos lymphoma cells following macrophage pre-treatment with goat polyclonal anti-PILRA antibodies (Cat #PA5-47613, Thermo Fisher). Pre-treatment of macrophages with anti-PILRA antibodies dampened the ability of SMAGP-overexpressing lymphoma cells to suppress phagocytosis, indicating that blockade of macrophage PILRA with antibodies is sufficient to partially block the ability of cancer-cell SMAGP to inhibit phagocytosis (FIGS. 7 and 11A). Survival analysis from the clinical data of the TCGA dataset Pan-Cancer Atlas indicated that higher expression of SMAGP in certain cancer types was related to worse patient outcomes (FIG. 11C), with three of them indicating statistical significance (FIGS. 15A-15C), supporting that SMAGP-PILRa can work as a potential target to improve cancer treatment. What makes SMAGP a potential ideal target in cancer treatment is that, SMAGP is not expressed in high levels across different tissue types in the human body (FIG. 15D), compared to the expression of CD47 (FIG. 15E) and HER2 (FIG. 15F). The expression level of SMAGP in whole blood is similar to that of HER2, and much lower than that of CD47, both used as targets against cancer.

### Example 6

### Mutation of SMAGP Glycosylation Sites Affects Binding to PILRa and SMAGP-Mediated Phagocytosis Suppression

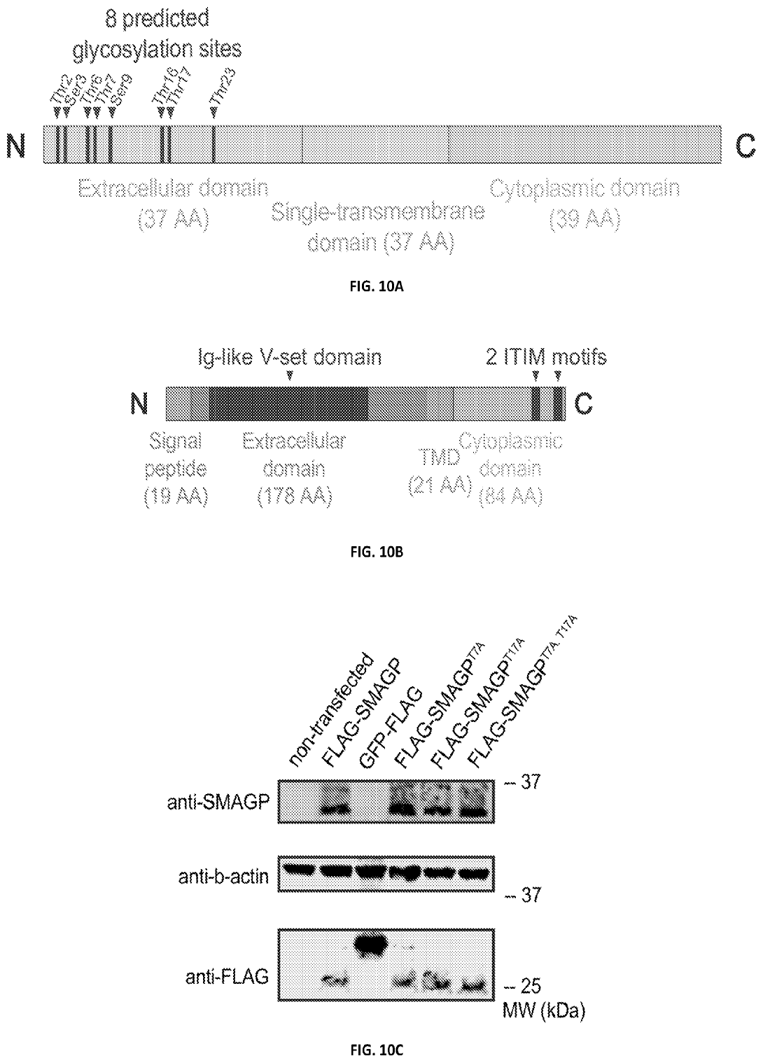

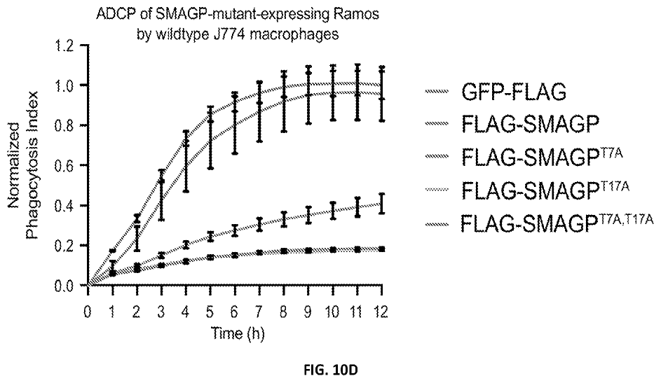

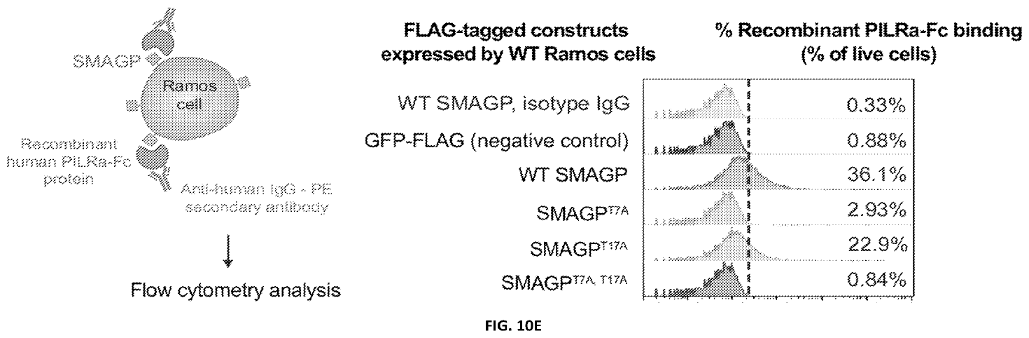

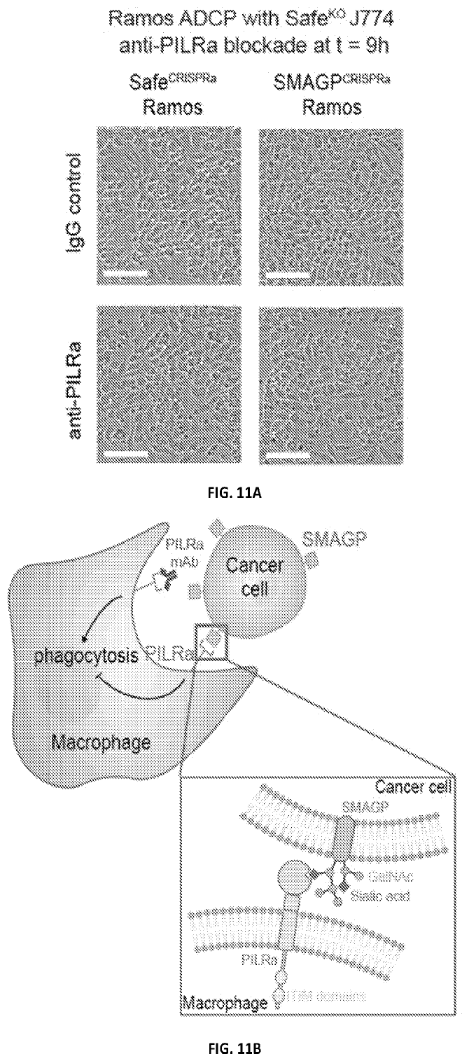

While the data indicated that PILRa facilitates SMAGP protection against ADCP, the mechanism by which SMAGP communicates with PILRa as an anti-phagocytic factor is still unknown. SMAGP has eight predicted glycosylation sites on its extracellular domain (FIG. 10A), and SMAGP is modified by both O-linked oligosaccharides and sialic acids. At the same time, PILRa crystal structure revealed that it adopts a typical siglec (sialic acid-binding immunoglobulin-like lectin) fold structure that can bind to sTn antigens (FIG. 10B).

Closer study of PILRa crystal structure during binding to known sTn ligands indicated that proline residues participate in the binding, and that the motifs sTn-TPxP or sTn-TPxxP are conserved among the determined or predicted recognition sites of PILRa ligands. In search of similar motifs in SMAGP extracellular domain, it was hypothesized that SMAGP^{Thr7} and SMAGP^{Thr17} are involved in PILRa binding as they match the conserved PILRa binding motifs, while SMAGP^{Ser9} and SMAGP^{Ser27} may play roles as well due to proline residue at +1 positions.

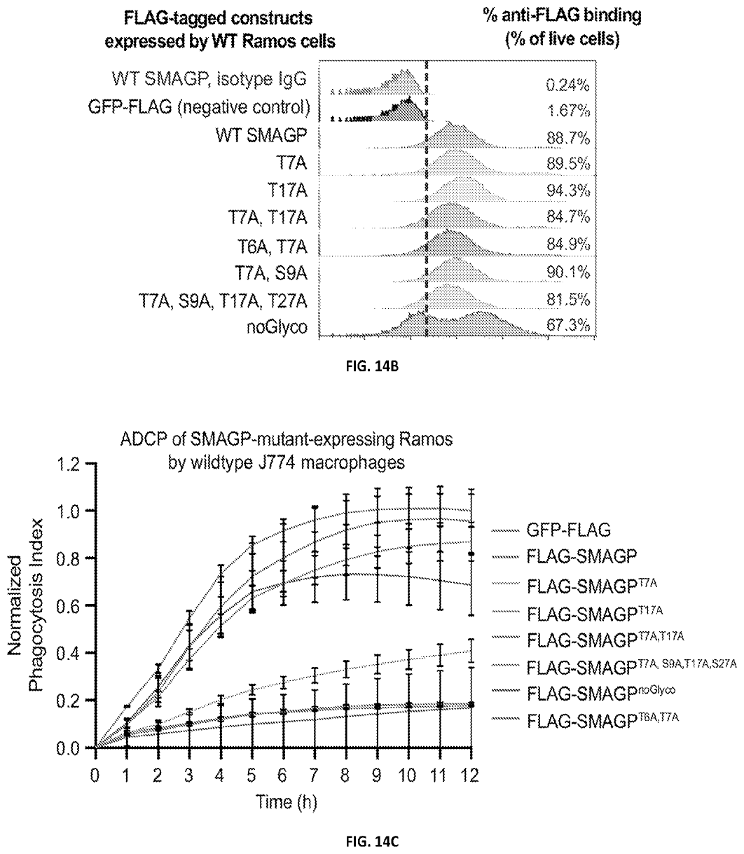

To decipher the key glycosylation sites on SMAGP for PILRa binding, Ramos cell lines that express N-terminus FLAG-tagged wild-type SMAGP or mutant SMAGP that has single, double, quadruple, or all glycosylation sites mutated to alanine were generated. Ramos cells expressing GFP-FLAG construct were used as negative control cells. Stable expressing lines were generated first based on blasticidin marker selection, then by Flag-tag sorting. SMAGP mutants were expressed at similar levels as wild-type SMAGP, as determined by Western blot (FIGS. 10C and 14A), and surface expression of wild-type and mutant SMAGP were at similar levels, as determined by flow cytometry (FIG. 14B). Then, the Ramos cells were co-incubated with J774 macrophages to determine the minimum mutations which inhibit SMAGP function. SMAGP mutants with most single mutation and double mutations retained SMAGP function on reducing phagocytosis, while only SMAGP^{T7A} slightly increased phagocytosis. However, the protective effect SMAGP has against phagocytosis was nearly completely abolished when ^{Thr7} and ^{Thr17} were both mutated (FIGS. 10D and 14C). To examine whether SMAGP^{T7A,T17A} enhanced phagocytosis by disrupting the binding between SMAGP and PILRa, a binding assay was conducted with human recombinant PILRa-Fc protein, with an anti-human-IgG-PE secondary antibody as a readout (FIG. 10E). For Ramos cells overexpressing SMAGP^{WT}, 36.1% of binding to PILRa was observed, indicating direct binding between SMAGP and PILRa (FIG. 10E). As for single mutant SMAGP^{T17A} which retains the protective effect, a reasonable amount (22.9%) of binding to PILRa was observed. For double mutant SMAGP^{T7A,T17A}, which lost the protective effect that SMAGP^{WT} had, lost its ability to bind to PILRa. However, for the single mutant SMAGPT17A which only partially lost the protective function, resulted in nearly complete loss of binding to PILRa. Interestingly, for double mutants that involved T7A but not T17A (for example, SMAGP^{T6A,T7A}, SMAGP^{T7A,S9A}), though they both retained the protective function of SMAGP^{WT} (FIG. 14C), they unexpectedly completely lost the binding ability to PILRa (FIG. 14D). The observations suggest that SMAGP Thr7 functions in PILRa binding, yet Thr17 may also function in signal transduction to PILRa. Mutations of both Thr7 and Thr17 abolish the protective function of SMAGPWT to improve cancer cell phagocytosis.

It is understood that the foregoing detailed description and accompanying examples are merely illustrative and are not to be taken as limitations upon the scope of the disclosure, which is defined solely by the appended claims and their equivalents.

All publications and patents mentioned in the above specification are herein incorporated by reference as if expressly set forth herein. Various changes and modifications to the disclosed embodiments will be apparent to those skilled in the art and may be made without departing from the spirit and scope thereof.

What is claimed is:

1. A method of treating a disease or disorder comprising administering to a subject in need thereof an inhibitor of paired immunoglobulin-like type 2 receptor-alpha (PILRA).

2. The method of claim 1, further comprising administering an inhibitor of at least one anti-phagocytic factor.

3. The method of claim 1 or 2, wherein the at least one anti-phagocytic factor comprises small cell adhesion glycoprotein (SMAGP).

4. The method of any of claims 1-3, further comprising administering at least one or both of a tumor antigen (TA)-targeting antibody and a CD47 blocking antibody.

5. The method of claim 4, wherein the TA-targeting antibody is a CD20 blocking antibody.

6. The method of claim 4 or 5, wherein the CD47 blocking antibody comprises an anti-CD47 antibody, an anti-SIRPalpha antibody, or any combination thereof.

7. The method of any of claims 1-6, wherein the disease or disorder comprises an autoimmune disorder, cancer, or atherosclerosis.

8. The method of claim 7, wherein the cancer is resistant to antibody-dependent cellular phagocytosis (ADCP).

9. The method of claim 7 or 8, wherein the cancer overexpresses SMAGP.

10. The method of any of claims 7-9, wherein the cancer is a solid tumor.

11. The method of any of claims 7-10, wherein the cancer comprises lymphoma, cervical cancer, lung cancer, colorectal cancer, ovarian cancer, breast cancer and/or leukemia.

12. The method of any of claims 7-11, wherein the autoimmune disorder comprises rheumatoid arthritis or multiple sclerosis.

13. A method of enhancing phagocytosis of a target cell comprising contacting an immune cell with an inhibitor of paired immunoglobulin-like type 2 receptor-alpha (PILRA).

14. The method of claim 13, wherein the immune cell comprises an innate immune cell.

15. The method of claim 14, wherein the innate immune cell is a monocyte, a neutrophil, or a macrophage.

16. The method of any of claims 13-15, wherein the target cell overexpresses SMAGP.

17. The method of any of claims 13-16, wherein the target cell is a cancer cell.

18. The method of claim 17, wherein the cancer cell comprises or is derived from lymphoma, cervical cancer, lung cancer, colorectal cancer, ovarian cancer, breast cancer and/or leukemia.

19. The method of any of claims 13-18, wherein contacting the immune cell comprises administration to a subject in need thereof.

20. A composition comprising an inhibitor of paired immunoglobulin-like type 2 receptor-alpha (PILRA) and an inhibitor of at least one anti-phagocytic factor.

21. The composition of claim 20, wherein the at least one anti-phagocytic factor comprises small cell adhesion glycoprotein (SMAGP).

22. The composition of claim 20 or 21, further comprising at least one or both of a tumor antigen (TA)-targeting antibody and a CD47 blocking antibody.

23. The composition of claim 22, wherein the TA-targeting antibody is a CD20 blocking antibody.

24. The composition of claim 22 or 23, wherein the CD47 blocking antibody comprises an anti-CD47 antibody, an anti-SIRPalpha antibody, or any combination thereof.

25. Use of a paired immunoglobulin-like type 2 receptor-alpha (PILRA) for treating a disease or disorder or enhancing phagocytosis of a target cell.
